(19) United States
(12) Patent Application Publication
(10) Pub. No.: US 2026/0248374 A1
(43) Pub. Date: Aug. 27, 2026

(54) INTRAORAL SCANNER WITH AN ANTENNA

(71) Applicant: 3SHAPE A/S, Copenhagen K (DK)

(72) Inventors: Michael PEDERSEN, Copenhagen K (DK); Jonas Sejr BORCH, Copenhagen K (DK)

(73) Assignee: 3SHAPE A/S, Copenhagen K (DK)

(21) Appl. No.: 18/874,754

(22) PCT Filed: Jun. 21, 2023
(86) PCT No.: PCT/EP2023/066749

§ 371 Date: Dec. 13, 2024

(30) Foreign Application Priority Data
Jun. 22, 2022 (EP) 22180500

(51) Int. Cl. A61B 1/24; A61B 1/00; A61B 1/05; A61B 1/06; H01Q 1/22; H01Q 9/04; H01Q 21/28

(52) U.S. Cl. CPC A61B 1/24; A61B 1/00016; A61B 1/00032; A61B 1/0004; A61B 1/05; A61B 1/0676; H01Q 1/2291; H01Q 9/04; H01Q 21/28; A61B 2560/0214

(57) ABSTRACT

Embodiments can relate to an intraoral scanner including a projector unit configured to emit light at least onto a dental object of a patient, and an image sensor configured to acquire reflected light from at least the dental object. The scanner includes a wireless interface configured to communicate with an external device, where the communication may include 2D image data and/or 3D image data processed or raw. The scanner includes a housing accommodating the projector unit and the image sensor, and wherein the housing includes a first end and a second end, and the projector unit and the image sensor are arranged closer to the first end, and an antenna of the wireless interface is arranged closer to the second end, and wherein during a scan of an oral cavity the first end is located inside the oral cavity and the second end is not located within the oral cavity.

### FIELD

The disclosure relates to wireless communication via an antenna. In particular, the disclosure relates to an antenna with improved performance and compactness.

### BACKGROUND

An intraoral scanner plays an important role in transforming both restorative and orthodontic dentistry. Real-time imaging using the intraoral scanner allows for creating three-dimensional digital model of single or multiple teeth, whole arches which may include restorations or implants, opposition arches, occlusion, and surrounding soft tissue or even dentures for edentulous patients. With on-screen display of the three-dimensional digital model, explaining treatment opportunities to patients is simplified. Patients appreciate the more comfortable data-acquisition process. Similarly, dental professionals appreciate the ease and efficiency of using the intraoral scanner. For improving the use of the intraoral scanner, a wireless interface is added to the scanner for allowing wireless communication of data between the scanner and an external device during the use of the scanner, and where the external device is configured to at least display the data. For improving even more, the useability of the scanner, the scanner is powered by a battery. Unfortunately, electromagnetic signals transferred to and from an antenna of the wireless interface is damped significantly when travelling through tissue, water and other anatomically elements part of the mouth. In relation to the wireless signal quality, it is very important that the antenna is arranged in or on the scanner such that the mouth of a patient or a hand of a user of the scanner does not apply any unwanted shadowing effect onto the antenna

### SUMMARY

An aspect of the present disclosure is to provide a solution to the problem of not creating any shadowing effect onto an antenna of an intraoral scanner that is cause by a mouth of a patient and/or a hand of a user of the scanner.

A further aspect of the present disclosure is to provide a compact intraoral scanner including an antenna that allows a more compact intraoral scanner than what is known today.

According to the aspects, an intraoral scanner is disclosed. The intraoral scanner comprising a projector unit configured to emit light at least onto a dental object of a patient, and an image sensor configured to acquire reflected light from at least the dental object. The projector unit is configured to emit white, light, visible light, none-visible light, UV light, and/or infrared light. The image sensor is configured to receive visible and none-visible light (e.g. 700 nm to 3000 nm). The scanner includes a wireless interface configured to communicate with an external device, where the communication may include 2D image data and/or 3D image data processed or raw. The scanner includes a housing that accommodates the projector unit and the image sensor, and wherein the housing includes a first end and a second end, and the projector unit and the image sensor are arranged closer to the first end, and an antenna of the wireless interface is arranged closer to the second end, and wherein during a scan of an oral cavity of a patient the first end is located inside the oral cavity and the second end is not located within the oral cavity. By arranging the antenna closer to the second end the shadowing effect that is caused by the mouth (i.e. the oral cavity) of the patient, or the hand of the operator is reduced. Thereby, an improved wireless signal quality of the wireless interface is obtained.

The antenna may be arranged within the housing for the purpose of improving the hygiene of the intraoral scanner. If the antenna was placed outside the housing, then is would be more difficult to maintain the level of hygiene of the intraoral scanner in order to be used as a medical device.

The intraoral scanner may be configured for determining 3D geometry and color of at least a part of the surface of an object in an oral cavity. The intraoral scanner may comprise at least one camera accommodating an array of sensor elements; a pattern generator configured to generate, using a light source, a probe light with a plurality of configurations in the form of a time-varying illumination pattern; an optical system for transmitting the probe light towards the object along an optical path thereby illuminating at least a part of the object with the time-varying illumination pattern, and for transmitting at least a part of the light returned from the object to the at least one camera to form a plurality of 2D images, wherein the 3D geometry is determined based on the plurality of 2D images and the time-varying illumination pattern; and a tip configured to be inserted into the oral cavity. Furthermore, the intraoral scanner may comprise a hardware processor configured to: selectively switch a color of the probe light, thereby illuminating the object with different colors at different times; record different images by the at least one camera at the different times, thereby recording images of the object with the different colors; and combine the different colors from the different images, thereby obtaining the color of the surface of the object, wherein the intraoral scanner is wireless, and wherein the at least one camera is a high-speed camera. The tip may be attached to the first end of the housing.

The antenna may be a dual band antenna configured to wireless communicate at around 2.4 and 5.8 GHz. The antenna may be a monopole antenna or a loop antenna. The antenna may include a ground plane and an active part, where the active part is the radiation part. The antenna may be a miniature surface mounted device (SMD) edge mounted ceramic antenna. The antenna may be configured to Bluetooth, Bluetooth low energy, and/or WIFI.

It is an advantage of providing a dual mode antenna configuration with same components, in that the size of the intraoral scanner may be reduced, while at the same time increasing the wireless capability.

The wireless interface may include at least two antennas, a first antenna configured to communicate via a WIFI protocol and a second antenna configured to communicate via a Bluetooth/Low energy protocol. The scanner is configured to switch between the two antennas depending on the availability and signal quality of the two antenna. For example, a WIFI signal quality may be poor in a part of a building where the scanner is located, and in this situation it will be more suitable to use the second antenna for communicating to an external device via the Bluetooth protocol (or Bluetooth Low energy protocol). In another example, the external device may not be able to communicate via a Bluetooth protocol, and thereby, the scanner automatically switch to the first antenna as no Bluetooth partner, i.e. an external device, is available for communication. The pairing between the external device and the scanner may be done automatically, as the scanner is configured to scan occasionally and automatically for external devices configured to communicate using Bluetooth. If the scanner detects an external device, an authentication of the external device is performed, and if the authentication succeed then the pairing between the scanner and the external device begins. The scanning for external devices performed by the scanner may be done in intervals of between 10 to 30 seconds, 30 to 60 seconds, 10 to 60 seconds, when no use of WIFI or if the WIFI signal is critically low. WIFI signal being critical low may result in a significant increase of packet loss in the WIFI communication link, and that may result in that a scanning of a patient's mouth would not be possible to carry out properly.

In another situation, the two antennas may both be configured to communicate via the WIFI protocol or Bluetooth protocol. The advantage of having two antennas configured to communicate with the same protocol is that if the bandwidth of a single WIFI antenna is not enough for streaming data from the scanner to the external device in real time, then the data to be streamed have to be divided into two groups of data where each of the two groups are being transmitted to the external device in separate antenna. In this situation, the data being communicated via both antennas must be synchronized in order not to erroneously change the order of data packages being communicated. The synchronization may be performed by the scanner (or the external device) which receives a synchronization signal from the external device via both antennas, and based on a timing of the synchronization signal arriving at the scanner, the scanner is configured to synchronize the communication via the two antennas. In another example, the external device determines the synchronization between the two antennas based on a synchronization signal being transmitted via both antennas to the external device. The external device detects a first receiving time for a first synchronization signal and a second receiving time for a second synchronization signal, and based on a time difference between the first and the second receiving time, the external device is configured to communicate a synchronization signal to the scanner, wherein the synchronization signal is then used by the scanner for synchronizing the communication via the two antennas.

The synchronization may be done automatically during booting of the scanner or based on a request by the user of the scanner. The request may be provided via a user interface of the scanner or the external device.

The housing may include a user interface arranged on an outer surface of the housing, and when the scanner is being used intentionally, the user will place his/her's hand on the outer surface of the housing and close to the user interface, such that a finger of the hand is able to interact with the user interface during the intentionally use. And therefore, for improving the range of communication of the antenna, then the antenna must be arranged close to the second end than to the user interface to avoid that the hand of the user is covering the antenna.

The antenna may be arranged on a first side of the user interface opposite to a second side of the user interface where the light is emitting outside the housing, and thereby, avoiding the antenna being placed inside the mouth of the patient during scanning. During the scanning of the patient's mouth, i.e. oral cavity, the hand of the user is placed on the first side of the user interface, and thereby, it is avoided that the part of the scanner being on the first side of the user interface will entering the mouth of the patient during scanning.

To make sure that an active part, i.e. radiation part, of the antenna is not being covered by the hand of the user, it may then be needed to place the active part of the antenna with a specific distance to the user interface or to the first end of the housing. For example, a distance between the user interface and a distal end of the antenna that is arranged closest to the second of the housing is within a range of 8 cm to 20 cm, 9 cm to 16 cm, 10 cm to 15 cm, 11 cm to 14 cm, about 8 cm, about 9 cm, about 10 cm, about 11 cm, about 12, about 13 cm about 14 cm, about 15 cm, or about 16 cm.

The intraoral scanner may include a battery or a battery package unit that can be detachably mounted to the scanner, and the battery package unit may include one or more batteries. The battery package unit may include a first battery end and a second battery end, and the first battery end is configured to be arranged closest to the first end of the housing, and a radiation part of the antenna is arranged closest to the second battery end. The battery package unit may be detachably mounted to the second end of the housing or about the second end of the housing. In some examples, the second battery end may be arranged outside the housing, but in this example, the second battery end is still located closest to the second end of the housing than the first end of the housing. In the examples, the antenna would not be covered by the hand during intentionally use of the scanner. The battery package unit may include a battery processor, wherein the battery processor is arranged closest to the first battery end. Thereby, the battery processor would not apply or at least limiting the electromagnetic noise to the antenna resulting in an improved antenna performance in comparison to placing the battery processor closer to the second battery end when the active part of the antenna is arranged closest to the second battery end.

The antenna may include a first sub-antenna part and a second sub-antenna part, and wherein the first sub-antenna part is arranged at a first side of the housing and the second sub-antenna part is arranged at a second side of the housing. Both sub-antenna parts are configured to radiate or receive an electromagnetic field. By placing the sub-antenna parts at the respective sides of the housing, the coverage of the antenna would not be affected by how the body of the user is positioned in relation to the scanner. The first sub-antenna part and the second sub-antenna part may be arranged symmetrically around a central longitudinal axis of the housing. The symmetrically arrangement of the sub-antenna parts provide a similar antenna performance irrespectively how the body of the user is positioned in relation to the scanner.

The first sub antenna and the second sub antenna may perform independently as antennas or may perform as one combined antenna.

The housing may include a central longitudinal axis, and the antenna may include a first sub-antenna part and a second sub-antenna part, and wherein the first sub-antenna part is arranged at a first side of the central longitudinal axis, and the second sub-antenna part is arranged at a second side of the central longitudinal axis.

The intraoral scanner includes a battery package unit, and the antenna includes a first sub-antenna part and a second sub-antenna part, and wherein the first sub-antenna part may be arranged at a first side of the battery package unit, and the second sub-antenna part may be arranged at a second side of the battery package unit. By placing the sub-antenna parts at the respective sides of the battery package unit, the coverage of the antenna would not be affected by how the body of the user is positioned in relation to the scanner with the battery package unit.

The housing may include a bottom surface that is opposite to an upper surface, and the antenna includes a first sub-antenna part and a second sub-antenna part, and both sub-antenna parts are arranged closest to the upper surface, and wherein the upper surface is longer than the bottom surface. Thereby, the distance from the sub-antenna parts to the user interface is maximized, and thereby, reducing the likelihood for the hand to cover the antenna fully or partially.

The antenna includes a first sub-antenna part and a second sub-antenna part, and both sub-antenna parts may have an electrical length between 1/16λ and λ. The antenna includes a first sub-antenna part and a second sub-antenna part, and both sub-antenna parts have a physical length of between 0.5 cm and 12.5 cm. The lengths of the antenna are suitable for the intraoral scanner.

An operating frequency of the antenna may be between 2.4 GHz and 8 GHz.

The antenna includes a first sub-antenna part and a second sub-antenna part, and both sub-antenna parts include a distal radiation end, and a physical length between the distal radiation ends is between 1 cm and 24 cm.

The antenna includes a first sub-antenna part and a second sub-antenna part, and both sub-antenna parts include the same type of antenna. The type of antenna is a monopole antenna, a dipole antenna, an IFA antenna or a PIFA antenna.

The antenna includes a first sub-antenna part and a second sub-antenna part, and both sub-antenna parts are connected to a transceiver of the intraoral scanner via a coaxial cable or a flex print. The wireless interface includes the transceiver. The flexible connection between the transceiver and the antenna allows for an easier way to arrange the antenna in the housing. Furthermore, the coaxial cable is shielded from electromagnetic noise interference that could reduce the quality of the wireless signal received by the antenna. The flex print may include an electromagnetic (EM) shielding layer that protects the connection wire(s) between the transceiver and the antenna from EM noise interference.

A first sub-antenna part and a second sub-antenna part of the antenna may be arranged on a printed circuit board, wherein each of the sub-antenna parts is printed onto or into a first surface of the printed circuit board, and wherein the first surface is directed away from a battery package unit of the intraoral scanner. Placement of the antenna within the housing is rather crucial for the performance of the antenna, and by forming the antenna by wires which are arranged freely within the housing may result in difference performance of the wireless interface between different scanners that include the wireless interface. To improve the stability of the antenna it would be of benefit to arrange the antenna on the printed circuit board as described above.

The wireless interface includes an LC matching network connected to the antenna, and wherein an operating frequency of the antenna is tunable by varying the values of one or more inductors and/or one or more capacitors of the LC matching network. The intraoral scanner is able to adapt the wireless interface to communicate with different communication protocols, and thereby, it is possible to provide a more compact intraoral scanner that is able to communicate with different communication protocols in comparison to an intraoral scanner including two antennas.

The antenna includes a first sub-antenna part and a second sub-antenna part, and wherein each of the sub-antenna parts includes a ground plane and a radiation part, and wherein the radiation part and the ground plane form a monopole antenna, and both sub antenna parts are arranged on a none-conductive material for the purpose of avoiding any unwanted degradation of the antenna performance.

The antenna includes a first sub-antenna part and a second sub-antenna part, and wherein each of the sub-antenna parts includes a ground plane and a radiation part, wherein the ground plane is arranged partly around the radiation part with a distance between the ground plane and the radiation part.

The antenna is arranged on an inner surface of the housing. The antenna may be mounted onto the inner surface or printed onto the inner surface providing a more reliable manufacturing process for placing the antenna within the housing.

The antenna may be arranged with a minimum distance of more than 8 mm or 10 mm to metal arranged within the housing, and thereby, the antenna performance is improved as shadowing effect of the antenna is reduced.

The antenna may be arranged at least partly external to the housing, and wherein the antenna may be arranged partly at the second end of the housing. The antenna may protrude away from the housing. At least a part of the antenna may protrude away from the housing, and another part may be arranged within the housing. The antenna is arranged more distinctly from any potential electromagnetic noisy components of the scanner, and that results in a more stable antenna performance.

The wireless interface may be configured to wireless communicate with an external device such as a computer, server, smartphone or a tablet and a display unit configured to display the data or images being communicated from the scanner. The external device may also contain a monitor to display the images or data processed into images. The patient is then able to see the images without the user of the scanner needs to move the display unit.

Thereby, it is possible to display simultaneously images from the scanner on the smartphone/tablet/computer and the display unit. The display unit may be a monitor that is connected to a computing device. The computing device may be a tablet, a laptop or any kind of a computer.

The intraoral scanner is configured to acquire intraoral scan data from a three-dimensional dental object during a scanning session. The intraoral scanner may comprise a multi-chip assembly including a plurality of integrated chips, and the plurality of integrated circuit chips including one or a combination of:
a power management chip,
a processor chip configured to process intraoral scan data of a patient and provide 2D image data and/or 3D image data, and
a wireless interface chip configured to transmit the 2D image data and/or the 3D image data,
   wherein the multi-chip assembly comprises:
a first layer having a surface,
a space layer being configured to accommodate one or more of the plurality of integrated circuit chips as one or more embedded chips,
a ground layer below the first layer and the spacer layer, and
a first shielding layer between the spacer layer and the first layer.

The intraoral scanner with embedded integrated circuit chips results in a significantly reduced size of the scanner, and thereby, makes it more comfortable for the patient to be scanned. Unfortunately, a more compact intraoral scanner places greater demands on shielding the antenna from EM noise.

The multi-chip assembly may be arranged within the intraoral scanner such that the first shielding layer is arranged between the battery and the antenna, or between an EM noisy component of the intraoral scanner and the antenna.

The ground layer may be electrically insulated from at least the first layer by means of an insulating layer. A connection to the ground layer may be provided via through-holes in the first layer, the spacer layer and the insulating layer, and additionally through any second and third layers. It is an advantage to provide the through-holes along one or more edges of the plurality of shielding layers to provide an optimum shielding of the components. In some embodiments, sufficient shielding may be obtained by providing the through-holes along edges of the components to be shielded.

The second layer may be the shielding layer. In some embodiments, the third layer may be the insulating layer. Thus, the spacer layer may be provided between second layer and the third layer, such as between the shielding layer and the insulating layer.

The multi-chip assembly may comprise a second layer and a third layer, and wherein the spacer layer is between a second layer and a third layer.

The intraoral scanner may comprise an insulating layer, wherein the spacer layer is between the shielding layer and the insulating layer.

The one or more embedded chips comprise the wireless interface chip and/or the power management chip. The wireless communication unit, or the wireless interface chip, is configured for wireless communication, including wireless data communication, including 2D images, 3D images, or raw images, and is interconnected with an antenna for emission and reception of an electromagnetic field or a magnetic field; the wireless interface chip comprises a transmitter, a receiver, a transmitter-receiver pair, such as a transceiver, a radio unit, etc. ; the wireless interface chip is configured for communication using any protocol as known for a person skilled in the art.

The wireless interface chip is based on 802.11a/b/g/n/ac/ad/af (WI-FI), Bluetooth/Bluetooth Low Energy (BLE), Zigbee, or Worldwide Interoperability for Microwave Access (WiMax) technology.

It is an advantage of embedding one or more chips in multi-chip assembly, in that noise from the multi-chip assembly may be reduced. It is an advantage of reducing noise from the multi-chip assembly without having to provide a so-called shielding can around the multi-chip assembly.

It is an advantage of one or more embodiments of the present disclosure that a reduction of noise and improvement of performance for antennas and the wireless communication systems as such, is enabled by embedding chips in the multi-chip assembly, e.g. using embedded die technology.

The multi-chip assembly may comprise a plurality of layers, such a 3 layer, 5 layers, such as at least 10 layers, such as up to 15 layers, such as 12 layers, such as between 10 and 15 layers. It is hereby possibly to shield embedded chips with multiple ground layers to decrease electromagnetic radiation, such as the E-field emitted, from the embedded chip, including e.g. from an embedded switching NP chip.

The intraoral scanner may comprise a magnetic induction antenna and wherein a first wireless interface chip is a magnetic induction control chip.

The intraoral scanner may be configured to communicate using magnetic induction, such a near-field magnetic induction. The magnetic induction control chip is an integrated circuit implementing magnetic induction transmit and receive functions, such as magnetic induction transmit and receive control functions. The magnetic induction control chip is interconnected to the magnetic induction antenna e.g. via electrical wires or via electrical conductive traces on a support substrate. The intraoral scanner may comprise the magnetic induction control chip and the magnetic induction antenna being configured to communicate using magnetic induction, such a near-field magnetic induction. The magnetic induction antenna may be a magnetic induction coil. The magnetic induction control chip may be configured to control power supply to the magnetic induction antenna.

The magnetic induction control chip may be configured to apply any modulation schemes including amplitude modulation, phase modulation, and/or frequency modulation to the data signal to be communicated via magnetic induction so that data are modulated onto the magnetic field emitted from the magnetic induction antenna. The magnetic induction control chip may comprise circuits, such as low noise amplifiers (LNA), mixers and filters. The magnetic induction control chip may also comprise peripheral digital blocks such as frequency dividers, codec blocks, demodulators, etc.

The magnetic induction antenna may be further configured for receiving a magnetic field communicated by another electronic device, such as via a magnetic induction antenna of another electronic device, and providing the received data signal to the magnetic induction control chip. The magnetic induction control chip may be configured to demodulate the received signal. In some embodiments the magnetic induction control chip is configured as a transceiver. In some embodiments, the magnetic induction control chip is configured to receive and transmit data at a particular frequency.

The data communicated may include data, settings, information, etc.

The magnetic induction antenna and the magnetic induction control chip may be configured to operate at a frequency below 100 MHz, such as at below 30 MHz, such as below 15 MHz, during use. The magnetic induction antenna may be configured to operate at a frequency range between 1 MHz and 100 MHz, such as between 1 MHz and 15 MHz, such as between 1 MHz and 30 MHz, such as between 5 MHz and 30 MHz, such as between 5 MHz and 15 MHz, such as between 10 MHz and 11 MHz, such as between 10.2 MHz and 11 MHz. The frequency may further include a range from 2 MHz to 30 MHz, such as from 2 MHz to 10 MHz, such as from 2 MHz to 10 MHz, such as from 5 MHz to 10 MHz, such as from 5 MHz to 7 MHz.

It is an advantage of using magnetic induction that typically low latency may be obtained. Especially when streaming image data, it is of importance to keep the latency low, to avoid delays noticeable by a user. Typically, a delay of less than 100 ms, such as of less than 50 ms, such as of less than 25 ms, such as of less than 10 ms, such as of less than 5 ms, such as of less than 1 ms, may be obtained by use of magnetic induction for communication.

By embedding the magnetic induction control chip in the multi-chip assembly and providing a shielding layer in the multi-chip assembly to shield between the magnetic induction control chip and a magnetic induction antenna provided at the surface of the first layer of the multi-chip assembly enables a compact design, with reduced interference between the magnetic induction antenna and the magnetic induction control chip. It is an advantage of providing a compact design as this will further reduce the amount of wires in the intraoral scanner which may further reduce electromagnetic interference, and thus may improve the overall EMC properties of the intraoral scanner.

However, it is envisaged that the intraoral scanner as herein disclosed is not limited to operation in such a frequency band, and the intraoral scanner may be configured for operation in any frequency band.

The at least one embedded chip includes a controlling chip, such as an integrated circuit chip configured to control the operation and/or the power supply to another component, such another component not provided in the multi-chip assembly.

The chip may be an electronic component. In some embodiments, the embedded chip is an embedded electronic component. In some embodiments, the embedded chip is an active device or an active electronic component.

The embedded chip may be a shielded embedded chip, such as an electromagnetically shielded embedded chip. The shielding provided by the plurality of shielding layers, including the first shielding layer.

The first shielding layer provides a shielding of the embedded electronic component. The ground layer may provide a shielding of the embedded electronic component. The first shielding layer and the ground layer may in combination provide a shielding of the embedded electronic component.

The first shielding layer may be configured to reduce electromagnetic emission from the one or more embedded chips.

The first shielding layer may be above one of the one or more embedded chips, and wherein the intraoral scanner further comprises a second shielding layer below the one or more embedded chips.

The one or more embedded chips comprise a first embedded chip and a second embedded chip, and wherein the intraoral scanner further comprises a shielding between the first embedded chip and the second embedded chip.

The shielding may be in the spacer layer.

The first embedded chip may be the power management chip and the second embedded chip may be the wireless interface chip.

The intraoral scanner may comprise a magnetic induction antenna, wherein the wireless interface chip is a magnetic induction control chip.

The magnetic induction control chip may be one of the one or more embedded chips, and wherein the magnetic induction antenna may be at the surface of the first layer; and wherein the first shielding layer and/or another shielding layer provides a shield between the magnetic induction control chip and the magnetic induction antenna.

The plurality of integrated circuit chips also includes an additional wireless interface chip. The additional wireless interface chip may be a RF wireless interface chip, and wherein the intraoral scanner further comprises a RF antenna.

It is an advantage that by one or more embodiments as presented, an RF antenna and a magnetic induction antenna may be provided in the intraoral scanner. To have an RF antenna and a magnetic induction antenna provided in the intraoral scanner increases the wireless communication capabilities of the intraoral scanner. However, providing both an RF antenna and a magnetic induction antenna within a intraoral scanner, with the restrictions as set out above pertaining to size, noise, EMC regulations, etc. has typically led to an increased size of the intraoral scanner to obtain the improved communication capabilities.

Furthermore, in present day communication systems, numerous different communication systems communicate at or about 2.4 GHz, 5 GHz, 5.8 GHz, and/or 60 GHz, and thus there is also a significant environmental electromagnetic noise in the frequency range at or about 2.4 GHz, 5 GHz, 5.8 GHz, and/or 60 GHz. It is an advantage that for some applications for which the noise may be acceptable, for example for data communication, an RF antenna may be used. For other applications, in which a high noise level may impact the transmission significantly, a magnetic induction antenna may be used.

The first shielding layer comprises through-holes along one or more edges of the first shielding layer.

The electronic component(s) comprises resistor(s), capacitor(s), inductor(s), transducer(s), diode(s), or a combination of the foregoing.

The plurality of integrated circuit chips may include a rechargeable battery regulator, and wherein the one or more embedded chips comprise the rechargeable battery regulator.

The intraoral scanner may comprise a battery.

The battery may be a rechargeable battery. The intraoral scanner further comprises a re-chargeable battery regulator. In some embodiments, the rechargeable battery regulator is provided as an embedded chip. In some embodiments, a first and a second re-chargeable batter regulator is provided e.g. to obtain a desired voltage supply, such as a voltage supply being of the size intended for the power supply for the multi-chip assembly. In some embodiments, the re-chargeable battery supplies a power that is regulated down for the voltage supply to multi-chip assembly, and possibly further electronic components of the intraoral scanner. In some embodiments, a first regulator is used for regulating the power from a first voltage level to a lower voltage level, and a second regulator is configured for regulating the power from a second voltage level, being lower than the first voltage level, to a lower voltage level. In a further example, a third regulator is used for regulating the power from a third voltage level to a higher voltage level. The scanner may include one or more of the regulators. Any power conversion provides noise which influences the overall signal to noise ratio of the intraoral scanner. It is therefore an advantage of being able to embed one or more of the rechargeable battery regulators in the multi-chip assembly to reduce noise emitted from the rechargeable battery regulator(s).

It is an advantage of the present disclosure, that the battery does not need to provide a shielding function between the magnetic induction control chip and/or a power management circuits. In some embodiments, the magnetic induction control chip and the power management circuit are provided at a first side of the battery, wherein the magnetic induction antenna is provided at the same first side of the battery. A further electromagnetic shield may be provided between the magnetic induction antenna and the magnetic induction control chip, for example in the form of an encapsulation of the magnetic induction antenna, or in the form of a shield formed by a carrier, such as a carrier carrying intraoral scanner components, such as electronic components of the intraoral scanner.

The intraoral scanning device may be configured to acquire intraoral scan data from a three-dimensional dental object during a scanning session. The intraoral scanning device may comprise a circuit comprising a wireless communication unit, wherein the wireless communication unit is configured for wireless communication, a battery package unit configured to supply power for the intraoral scanner, wherein the circuit may be coupled to a positive pole and a negative pole of the battery package unit; wherein the battery package unit may be configured for electromagnetic field emission and electromagnetic field reception; wherein the battery package unit may be configured to oscillate at a frequency controlled by the circuit; and wherein the battery package unit comprises a battery that may be configured to operate as a battery antenna or the battery package unit comprises an antenna.

The battery package unit is configured to be attachable connected to the scanner, and the battery within the battery package unit is rechargeable. The advantage of arranging the antenna within the battery package unit is that an easy replacement of the antenna is possible if the antenna is defect. Furthermore, it would also be easier to change the antenna to a different type of an antenna, for example from a WIFI configured antenna to a 60 GHz configured antenna, or from a WIFI configured antenna to a Bluetooth configured antenna.

The first shielding layer may be arranged between the battery and the antenna.

The circuit may be coupled to the battery via multiple transmission lines that provide battery-feed signals, and wherein the circuit may be configured to cause the battery to oscillate at the frequency or the antenna arranged within the battery package unit.

The battery may be configured for electromagnetic field emission and electromagnetic field reception, and wherein the circuit may be configured to prevent the power management circuit from receiving battery power from the battery at frequencies above a threshold; and wherein the circuit may be coupled to both a positive pole and a negative pole of the battery, and wherein the circuit is configured to provide battery-feed signals to the battery via multiple transmission lines.

The circuit may be configured to allow battery power from the battery to be delivered to the power management circuit at frequencies below a threshold; and wherein the circuit is coupled to both a positive pole and a negative pole of the battery, and wherein the circuit is configured to provide battery-feed signals to the battery via multiple transmission lines. The battery may be configured to operate at a frequency controlled by the circuit; and wherein the circuit that is coupled to the positive and negative poles of the battery is configured to provide battery-feed signals to the battery via multiple transmission lines.

The circuit comprises one or more tuning components configured to determine an impedance.

The circuit comprises one or more tuning components configured to tune an impedance with respect to a wavelength of an electromagnetic field emitted or received by the battery.

The circuit comprises one or more tuning components, the one or more tuning components comprising an inductor, a capacitor, a tuning transmission line, or any combination thereof.

The tuning transmission line comprises a quarter wavelength transmission line.

The circuit comprises one or more tuning components having an inductive reactance that is between ½ nH and 50 nH.

The circuit comprises one or more tuning components having a capacitive reactance that is between 0.1 pF and 100 pF.

The circuit comprises one or more tuning components having an RF impedance magnitude that is at least 100 Ohm.

The intraoral scanner may comprise one or more parasitic antenna elements, wherein at least one of the one or more parasitic antenna elements has a free end. The parasitic antenna element is inductively connected to the antenna. The advantage of the parasitic antenna element is that the bandwidth of the antenna is improved significantly. Thereby, more data, i.e. larger sized images, high frame rate or high bit rate, can be wireless communicated from the scanner to an external device, such as a laptop, a tablet, a smartphone etc.

A distance between at least a part of the one or more parasitic antenna elements and the battery is below 1/40 of the wavelength.

The at least a part of the one or more parasitic antenna elements is a free end of the one or more parasitic antenna elements.

The at least a part of the one or more parasitic antenna elements is a center part of the one or more parasitic antenna elements.

At least one of the one or more parasitic antenna elements is a floating parasitic antenna element.

The circuit comprises one or more tuning components configured to improve a coupling between the battery and one or more parasitic antenna elements at a certain frequency.

The intraoral scanner may comprise a parasitic antenna element, wherein at least a part of the parasitic antenna element forms at least a part of a loop around the battery.

The battery maybe configured as a parasitic antenna element.

The circuit may be configured to obtain a resonance that corresponds to a wavelength or to an electromagnetic frequency of an electromagnetic field emitted or received by the battery. The threshold may be 3 MHz.

The battery may be configured to oscillate at a frequency controlled by the circuit that is coupled to the positive pole and the negative pole of the battery. The threshold may be 300 kHz.

The battery may be configured to oscillate at a frequency controlled by the circuit that is coupled to the positive pole and the negative pole of the battery.

The circuit may comprise a filter circuit.

The circuit may comprise an antenna matching circuit.

### BRIEF DESCRIPTION OF THE FIGURES

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIGS. 1A and 1B illustrate different examples of an intraoral scanner;

FIG. 2 illustrates an example of an intraoral scanner;

FIGS. 3A and 3B illustrate different examples of an intraoral scanner;

FIGS. 4A and 4B illustrate different examples of an intraoral scanner;

FIGS. 5A to 5E illustrate different examples of a multi-chip assembly in an intraoral scanner;

FIG. 6 illustrates an example of a multi-chip assembly in an intraoral scanner; and

FIGS. 7A and 7B illustrate different examples of an intraoral scanner.

### DETAILED DESCRIPTION

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the devices, systems, mediums, programs and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

The electronic hardware may include microprocessors, microcontrollers, digital signal processors (DSPs), field programmable gate arrays (FPGAs), programmable logic devices (PLDs), gated logic, discrete hardware circuits, and other suitable hardware configured to perform the various functionality described throughout this disclosure. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

A scanning for providing intra-oral scan data may be performed by a dental scanning system that may include an intraoral scanning device such as the TRIOS series scanners from 3Shape A/S. The dental scanning system may include a wireless capability as provided by a wireless network unit. The scanning device may employ a scanning principle such as triangulation-based scanning, confocal scanning, focus scanning, ultrasound scanning, x-ray scanning, stereo vision, structure from motion, optical coherent tomography OCT, or any other scanning principle. In an embodiment, the scanning device is operated by projecting a pattern and translating a focus plane along an optical axis of the scanning device and capturing a plurality of 2D images at different focus plane positions such that each series of captured 2D images corresponding to each focus plane forms a stack of 2D images. The acquired 2D images are also referred to herein as raw 2D images, wherein raw in this context means that the images have not been subject to image processing. The focus plane position is preferably shifted along the optical axis of the scanning system, such that 2D images captured at a number of focus plane positions along the optical axis form said stack of 2D images (also referred to herein as a sub-scan) for a given view of the object, i.e. for a given arrangement of the scanning system relative to the object. After moving the scanning device relative to the object or imaging the object at a different view, a new stack of 2D images for that view may be captured. The focus plane position may be varied by means of at least one focus element, e.g., a moving focus lens. The scanning device is generally moved and angled during a scanning session, such that at least some sets of sub-scans overlap at least partially, in order to enable stitching in the post-processing. The result of stitching is the digital 3D representation of a surface larger than that which can be captured by a single sub-scan, i.e. which is larger than the field of view of the 3D scanning device. Stitching, also known as registration, works by identifying overlapping regions of 3D surface in various sub-scans and transforming sub-scans to a common coordinate system such that the overlapping regions match, finally yielding the digital 3D model. An Iterative Closest Point (ICP) algorithm may be used for this purpose. Another example of a scanning device is a triangulation scanner, where a time varying pattern is projected onto the dental object and a sequence of images of the different pattern configurations are acquired by one or more cameras located at an angle relative to the projector unit.

The scanning device comprises one or more light projectors configured to generate an illumination pattern to be projected on a three-dimensional dental object during a scanning session. The light projector(s) preferably comprises a light source, a mask having a spatial pattern, and one or more lenses such as collimation lenses or projection lenses. The light source may be configured to generate light of a single wavelength or a combination of wavelengths (mono-or polychromatic). The combination of wavelengths may be produced by using a light source configured to produce light (such as white light) comprising different wavelengths. Alternatively, the light projector(s) may comprise multiple light sources such as LEDs individually producing light of different wavelengths (such as red, green, and blue) that may be combined to form light comprising the different wavelengths. Thus, the light produced by the light source may be defined by a wavelength defining a specific color, or a range of different wavelengths defining a combination of colors such as white light. In an embodiment, the scanning device comprises a light source configured for exciting fluorescent material of the teeth to obtain fluorescence data from the dental object. Such a light source may be configured to produce a narrow range of wavelengths. In another embodiment, the light from the light source is infrared (IR) light, which is capable of penetrating dental tissue. The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental object is patterned. The back-lit mask projector may comprise a collimation lens for collimating the light from the light source, said collimation lens being placed between the light source and the mask. The mask may have a checkerboard pattern, such that the generated illumination pattern is a checkerboard pattern. Alternatively, the mask may feature other patterns such as lines or dots, etc.

The scanning device preferably further comprises optical components for directing the light from the light source to the surface of the dental object. The specific arrangement of the optical components depends on whether the scanning device is a focus scanning apparatus, a scanning device using triangulation, or any other type of scanning device. A focus scanning apparatus is further described in EP 2 442 720 B1 by the same applicant, which is incorporated herein in its entirety.

The light reflected from the dental object in response to the illumination of the dental object is directed, using optical components of the scanning device, towards the image sensor(s). The image sensor(s) are configured to generate a plurality of images based on the incoming light received from the illuminated dental object. The image sensor may be a high-speed image sensor such as an image sensor configured for acquiring images with exposures of less than 1/1000 second or frame rates in excess of 250 frames pr. second (fps). As an example, the image sensor may be a rolling shutter (CCD) or global shutter sensor (CMOS). The image sensor(s) may be a monochrome sensor including a color filter array such as a Bayer filter and/or additional filters that may be configured to substantially remove one or more color components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

The network unit may be configured to connect the dental scanning system to a network comprising a plurality of network elements including at least one network element configured to receive the processed data. The network unit may include a wireless network unit or a wired network unit. The wireless network unit is configured to wirelessly connect the dental scanning system to the network comprising the plurality of network elements including the at least one network element configured to receive the processed data. The wired network unit is configured to establish a wired connection between the dental scanning system and the network comprising the plurality of network elements including the at least one network element configured to receive the processed data.

The dental scanning system preferably further comprises a processor configured to generate scan data (such as intra-oral scan data) by processing the two-dimensional (2D) images acquired by the scanning device. The processor may be part of the scanning device. As an example, the processor may comprise a Field-programmable gate array (FPGA) and/or an Advanced RISC Machines (ARM) processor located on the scanning device. The scan data comprises information relating to the three-dimensional dental object. The scan data may comprise any of: 2D images, 3D point clouds, depth data, texture data, intensity data, color data, and/or combinations thereof. As an example, the scan data may comprise one or more point clouds, wherein each point cloud comprises a set of 3D points describing the three-dimensional dental object. As another example, the scan data may comprise images, each image comprising image data e.g. described by image coordinates and a timestamp (x, y, t), wherein depth information can be inferred from the timestamp. The image sensor(s) of the scanning device may acquire a plurality of raw 2D images of the dental object in response to illuminating said object using the one or more light projectors. The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp. Accordingly, the output of the processor is the scan data, and the scan data may comprise image data and/or depth data, e.g. described by image coordinates and a timestamp (x, y, t) or alternatively described as (x, y, z). The scanning device may be configured to transmit other types of data in addition to the scan data. Examples of data include 3D information, texture information such as infra-red (IR) images, fluorescence images, reflectance color images, x-ray images, and/or combinations thereof.

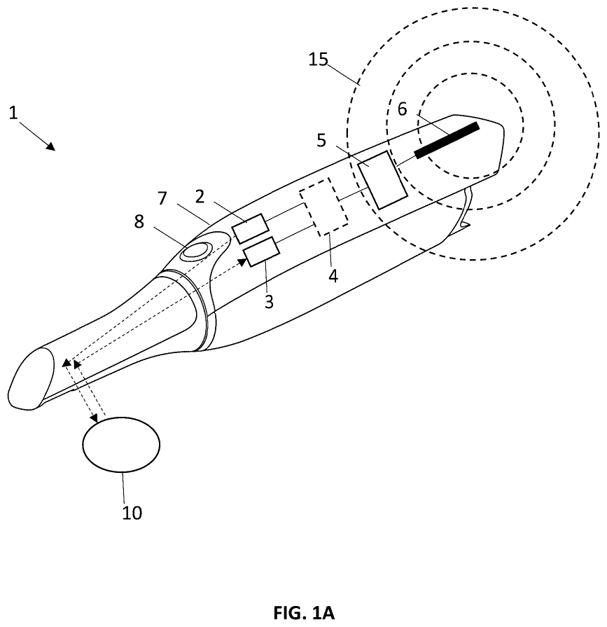

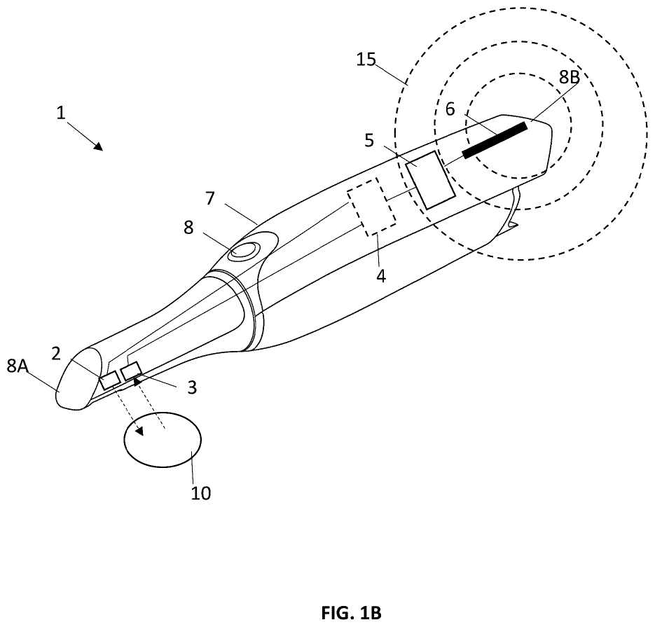

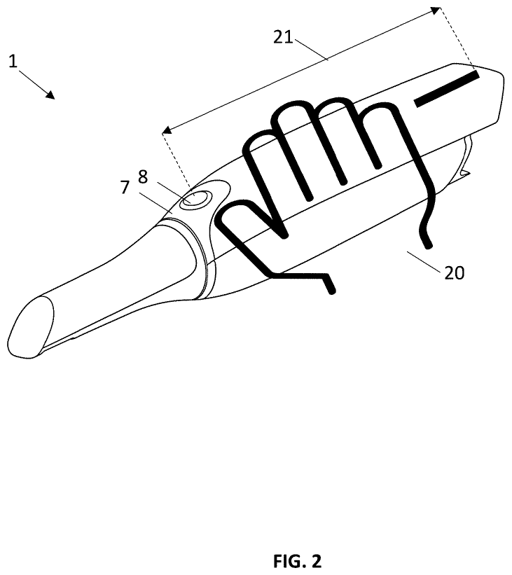

FIGS. 1A and 1B illustrate an intraoral scanner 1 that includes a projector unit 2, an image sensor 3, a processing unit 4, a wireless interface 5 that includes an antenna 6, and a housing 7 that accommodates in this example all above mentioned components of the intraoral scanner. The projector unit 2 is configured to emit light at least onto a dental object 10 of a patient, and the image sensor 3 is configured to acquire reflected light from at least the dental object 10. The wireless interface is configured to communicate 15 wirelessly with an external device, and in these present examples, the housing 7 accommodates the projector unit 2, the image sensor 3, the wireless interface 5, the antenna 6 and the processing unit 4. The housing includes a first end 8A and a second end 8B. The projector unit 2 and the image sensor 3 are arranged closer to the first end 8A than the second end 8B, and the antenna 6 of the wireless interface 5 is arranged closer to the second end 8B than the first end 8A. In FIG. 1A, the projector unit 2 and the image sensor 3 is arranged within a main body of the housing 7, and where FIG. 1B illustrates an example where the projector 2 unit and the image sensor 3 are arranged within a tip of the housing 7.

An outer surface of the housing 7 includes a user interface 8. In this present example, the user interface includes a single button. In other examples, the user interface 8 may include multiple buttons, a touch sensitive field, or any means configured to interact with a user of the scanner for controlling the scanner 1. In the present examples, the antenna 6 is arranged on a first side of the user interface 8 opposite to a second side of the user interface 8 where the light is emitting outside the housing 7.

The antenna has an operating frequency between 2.4 GHz and 8 GHz or between 2.4 GHz and 60 GHz. The type of antenna is a monopole antenna, a dipole antenna, an IFA antenna or a PIF A antenna.

The intraoral scanner 1 is configured to be handled by a single hand 20, and in order to make sure that the hand 20 is not covering the antenna while being on the housing and in vicinity to the user interface, it is then important to design the scanner housing to have a certain distance 21 between the antenna and the user interface. The distance between the user interface and a distal end of the antenna that is arranged closest to the second of the housing shall be within a range of 8 cm to 20 cm, 9 cm to 16 cm, 10 cm to 15 cm, 11 cm to 14 cm, about 8 cm, about 9 cm, about 10 cm, about 11 cm, about 12, about 13 cm about 14 cm, about 15 cm, or about 16 cm.

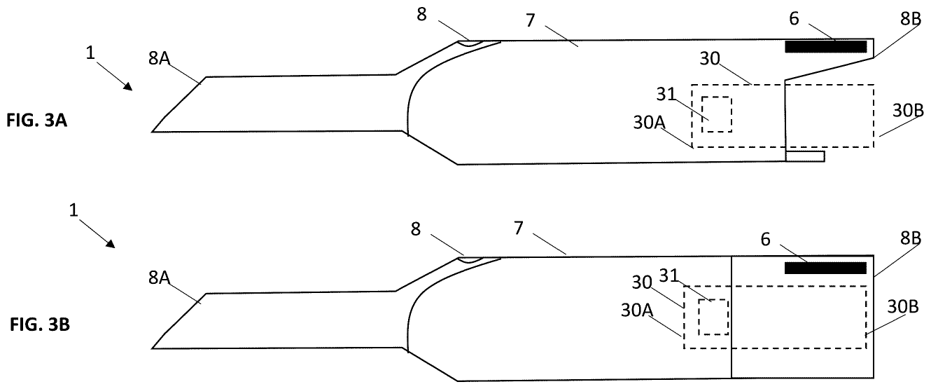

FIGS. 3A and 3B illustrates the intraoral scanner 1 from the side. In these example, the intraoral scanner 1 includes a battery package unit 30 that includes a first battery end 30A and a second battery end 30B, and the first battery end 30A is configured to be arranged closest to the first end 8A of the housing, and a radiation part of the antenna 6 is arranged closest to the second battery end. The battery package unit 30 may includes a battery processor for controlling the battery and LEDs arranged on a surface of the battery package unit 30. In FIG. 3A, the battery package unit is extending outside the housing 7, and in FIG. 3B, the battery package unit 30 is not extending outside the housing 7.

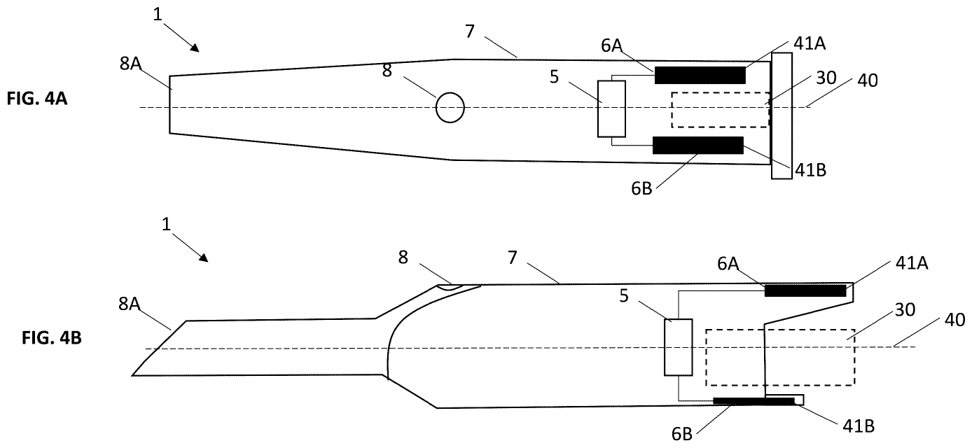

FIGS. 4A and 4B illustrates the intraoral scanner 1 from two different perspectives. FIG. 4A illustrates the intraoral scanner 1 from top, and FIG. 4B illustrates the intraoral scanner 1 from the side. On both FIGS, the antenna 6 includes a first sub-antenna part 6A and a second sub-antenna part 6B. The first sub-antenna part 6A is arranged at a first side of the housing 7 and the second sub-antenna part 6B is arranged at a second side of the housing 7. The two sub antennas (6A,6B) may be arranged symmetrically in the housing 7. In this specific example, the scanner 1 includes a battery package unit 30, and wherein the first sub-antenna part 6A is arranged at a first side of the battery package unit 30, and the second sub-antenna part 6B is arranged at a second side of the battery package unit 30. The housing 7 includes a central longitudinal axis 40, and wherein the first sub-antenna part 6A is arranged at a first side of the central longitudinal axis 40, and the second sub-antenna part 6B is arranged at a second side of the central longitudinal axis 40. In FIG. 4A the sub antennas are arranged symmetrically around the longitudinal axis in a horizontal direction, and in FIG. 4B, the sub antennas are arranged symmetrically around the longitudinal axis in a vertical direction. Both solutions provide a symmetrical antenna coverage in either direction in relation to the scanner. That minimizes the possibility of having a so called “dead zone” in the antenna coverage where the wireless communication quality is not suitable for transmitting 2D and/or 3D data in real time. The first sub-antenna part 6A and the second sub-antenna part 6B include a distal radiation end (41A,41B), and a physical length between the distal radiation ends is between 1 cm and 24 cm. The first sub-antenna part 6A and the second sub-antenna part 6B include the same type of antenna. The type of antenna is a monopole antenna, a dipole antenna, an IFA antenna or a PIFA antenna.

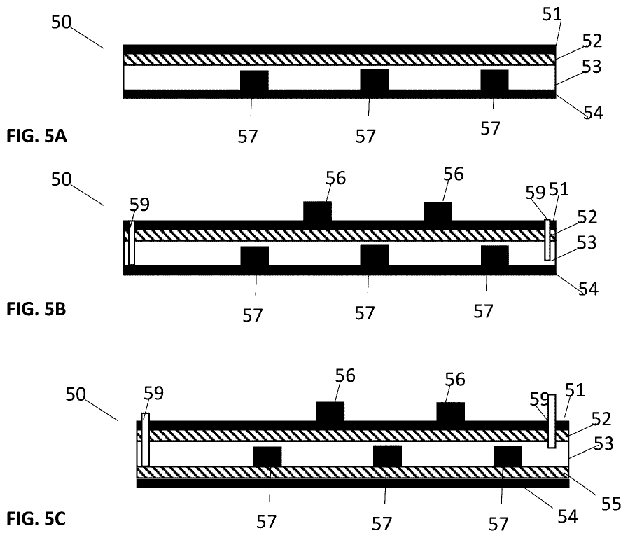

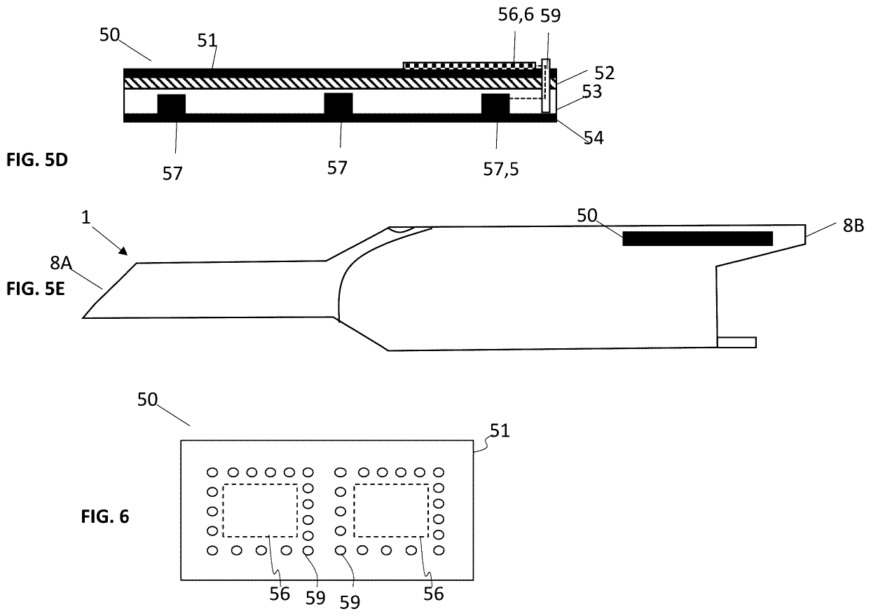

FIGS. 5A, 5B, 5C, and 5D illustrate an example of a multi-chip assembly 50 that includes embedded chip components 57, such as a power management chip 57, a processor chip 57 and a wireless interface chip 57, i.e. the wireless interface. The intraoral scanner 1 includes the multi-chip assembly 50. The multi-chip assembly 50 includes a first layer 51 having a surface, a space layer 53 being configured to accommodate one or more of the plurality of integrated circuit chips 57 as one or more embedded chips, a ground layer 54 below the first layer 51 and the spacer layer 53, and a first shielding layer 52 between the spacer layer 53 and the first layer 51.

The processor chip 57 is configured to process intraoral scan data of a patient and provide 2D image data and/or 3D image data. The wireless interface chip 57 is configured to transmit the 2D image data and/or the 3D image data to an external device. Furthermore, the wireless interface chip 57 is configured to communicate settings, data etc. The multi-chip assembly 50 may be arranged within the intraoral scanner 1 such that the first shielding layer is arranged between the battery and the antenna, or between an EM noisy component of the intraoral scanner 1 and the antenna (6,6A,6B).

FIGS. 5B and 5C illustrates through-holes 59 for allowing connection of surface chip components 56 applied to the first layer 51 to the ground layer 54. The surface chip component may for example be a power management chip, a processor chip, a wireless interface chip, a memory chip etc. In FIG. 5B the through-holes 59 is provided in the first layer 51, the shield layer 52, and the spacer layer 53, and in FIG. 5C, the through-holes 59 is provided in the first layer 51, the shield layer 52, the spacer layer 53 and the insulating layer 55. The though-holes 59 may be in additional layers, such as a second and a third layer. FIG. 5D illustrates the antenna 6 being mounted on the first surface 51 and a through-hole 59 that allows the antenna 6 to be connected to a transceiver of the wireless interface chip 56. For example, a wire may connect the antenna to the transceiver via the through-hole 59. FIG. 5E illustrates the intraoral scanner 1 comprising the multi-chip assembly 50. In this example, the assembly 50 is arranged at the second end of the housing 7.

FIG. 6 illustrates a top view of the multi-chip assembly 50 that at least includes two surface components 56 and multiple through-holes 59 arranged around each surface components 56.

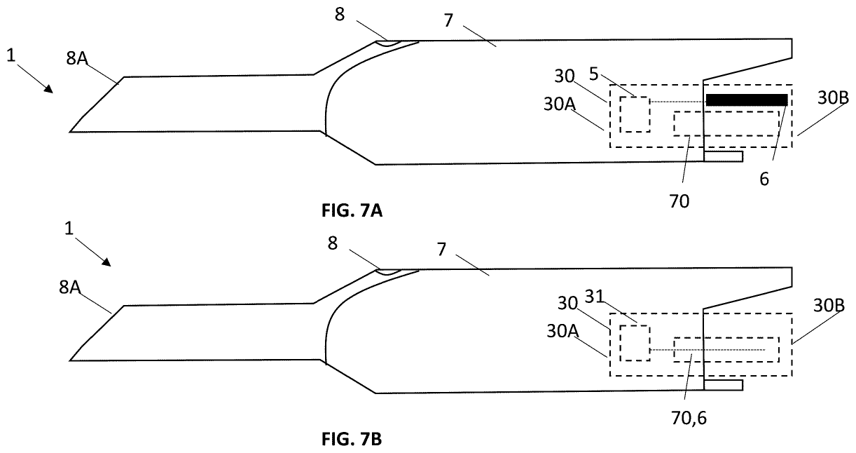

FIGS. 7A and 7B illustrate different examples of the antenna 6 being arranged within the battery package unit 30. The transceiver 5 of the wireless interface 5 is either arranged within the housing 7 or the battery package unit 30, but in this example the transceiver 5 is arranged within the battery package unit 30. In FIG. 7A the antenna 6 is separate from the battery 70, and in FIG. 7B the antenna 6 is the battery 70 or a battery cell 70. In both examples, the battery package unit 30 is configured for electromagnetic field emission and electromagnetic field reception. The battery package unit 30 is further configured to oscillate at a frequency controlled by the circuit. The battery package unit 30 is configured to be attachable connected to the scanner 1, and the battery 70 within the battery package unit 30 is rechargeable. The advantage of arranging the antenna 6 within the battery package unit 30 is that an easy replacement of the antenna 6 is possible if the antenna is defect. Furthermore, it would also be easier to change the antenna 6 to a different type of an antenna, for example from a WIFI configured antenna to a 60 GHz configured antenna, or from a WIFI configured antenna to a Bluetooth configured antenna.

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present invention.

Benefits, other advantages, and solutions to problems have been described herein with regard to specific embodiments. However, the benefits, advantages, solutions to problems, and any component(s)/ unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to an component/unit/element in the singular is not intended to mean “one and only one” unless explicitly so stated, but rather “one or more.” A claim may refer to any of the preceding claims, and “any” is understood to mean “any one or more” of the preceding claims.

It is intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e. to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element but an intervening elements may also be present, unless expressly stated otherwise. Furthermore, “connected” or “coupled” as used herein may include wirelessly connected or coupled. As used herein, the term “and/or” includes any and all combinations of one or more of the associated listed items. The steps of any disclosed method is not limited to the exact order stated herein, unless expressly stated otherwise.

It should be appreciated that reference throughout this specification to “one embodiment” or “an embodiment” or “an aspect” or features included as “may” means that a particular feature, structure or characteristic described in connection with the embodiment is included in at least one embodiment of the disclosure. Furthermore, the particular features, structures or characteristics may be combined as suitable in one or more embodiments of the disclosure. The previous description is provided to enable any person skilled in the art to practice the various aspects described herein. Various modifications to these aspects will be readily apparent to those skilled in the art, and the generic principles defined herein may be applied to other aspects.

The claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

1. An intraoral scanner comprising:
a projector unit configured to emit light at least onto a dental object of a patient;
an image sensor configured to acquire reflected light from at least the dental object;
a wireless interface configured to communicate with an external device, and
a housing that accommodates the projector unit and the image sensor, and wherein the housing includes a first end and a second end, and the projector unit and the image sensor are arranged closer to the first end, and an antenna of the wireless interface is arranged closer to the second end, and wherein the antenna is arranged within the housing, and
wherein during a scan of an oral cavity of a patient the first end is located inside the oral cavity and the second end is not located within the oral cavity.

2. The intraoral scanner according to claim 1, wherein the housing comprises an outer surface that includes a user interface, and where the antenna is arranged on a first side of the user interface opposite to a second side of the user interface where the light is emitting outside the housing.

3. The intraoral scanner according to claim 2, wherein a distance between the user interface and a distal end of the antenna that is arranged closest to the second of the housing is within a range of 8 cm to 20 cm, 9 cm to 16 cm, 10 cm to 15 cm, 11 cm to 14 cm, about 8 cm, about 9 cm, about 10 cm, about 11 cm, about 12, about 13 cm about 14 cm, about 15 cm, or about 16 cm.

4. The intraoral scanner according to claim 1, comprising a battery package unit that includes a first battery end and a second battery end, and the first battery end is configured to be arranged closest to the first end of the housing, and a radiation part of the antenna is arranged closest to the second battery end.

5. The intraoral scanner according to claim 4, wherein a battery processor of the battery package unit is arranged closest to the first battery end.

6. The intraoral scanner according to claim 1, wherein the antenna includes a first sub-antenna part and a second sub-antenna part, and wherein the first sub-antenna part is arranged at a first side of the housing and the second sub-antenna part is arranged at a second side of the housing.

7. The intraoral scanner according to claim 4, wherein the first sub-antenna part is arranged at a first side of the battery package unit, and the second sub-antenna part is arranged at a second side of the battery package unit.

8. The intraoral scanner according to claim 6, wherein the housing includes a central longitudinal axis, and wherein the first sub-antenna part is arranged at a first side of the central longitudinal axis, and the second sub-antenna part is arranged at a second side of the central longitudinal axis.

9. The intraoral scanner according to claim 8, wherein the first sub-antenna part and the second sub-antenna part are arranged symmetrically around the central longitudinal axis.

10. The intraoral scanner according to claim 6, wherein the first sub-antenna part and the second sub-antenna part have an electrical length between 1/16λ and λ.

11. The intraoral scanner according to claim 6, wherein the first sub-antenna part and the second sub-antenna part have a physical length of between 0.5 cm and 12.5 cm.

12. The intraoral scanner according to claim 1, wherein an operating frequency of the antenna is between 2.4 GHz and 8 GHz.

13. The intraoral scanner according to claim 6, wherein the first sub-antenna part and the second sub-antenna part include a distal radiation end, and a physical length between the distal radiation ends is between 1 cm and 24 cm.

14. The intraoral scanner according to claim 6, wherein the first sub-antenna part and the second sub-antenna part include the same type of antenna.

15. The intraoral scanner according to claim 14, wherein the type of antenna is a monopole antenna, a dipole antenna, an IFA antenna or a PIFA antenna.
